(19) United States
(12) Patent Application Publication
(10) Pub. No.: US 2005/0182432 A1
(43) Pub. Date: Aug. 18, 2005

(54) Apparatus and methods for clearing obstructions from surgical cutting instruments

(76) Inventors: Gary S. Fanton, Portola Valley, CA (US); John E. Ashley, San Francisco, CA (US)

Correspondence Address: HOEKENDIJK & LYNCH, LLP, P.O. BOX 4787, BURLINGAME, CA, 94011-4787, US

(21) Appl. No.: 10/782,489

(22) Filed: Feb. 18, 2004

(51) Int. Cl. A61B 17/32; A61M 31/00

(52) U.S. Cl. 606/168; 606/167

(57) ABSTRACT

The present invention relates to apparatus and methods for facilitating removal of obstructions from a surgical cutting instruments during a surgical procedure. The apparatus of the present invention is configured to interrupt flow in aspiration tubing when obstructions are detected in the cutting instrument. The apparatus then causes compression of the aspiration tubing to flush fluid towards the cutting instrument, thereby unclogging the instrument in a fast and efficient manner without the need to remove the instrument from the surgical site.

### FIELD OF THE INVENTION

The present invention relates to the field of surgical arthroscopy, and more particularly, to apparatus and methods for facilitating removal of clogged particulate matter from a surgical cutting instrument during a surgical procedure.

### BACKGROUND OF THE INVENTION

Surgical cutting instruments such as mechanical shaving systems or microdebriders are well known for use in treating injured tissue in various bodily locations, such as joints. Many conventional cutting instruments operate by continuously rotating or by reciprocal rotation of a cutting edge. Such cutting instruments may be used in conjunction with the provision of irrigation fluid to the surgical site, and with the provision of a suction source to aspirate cut bodily tissue and irrigation fluid from the surgical site. The suction source also serves to draw tissue to the cutting edge before the tissue is debrided.

A common problem associated with conventional cutting instruments is clogging of the opening of the cutting edge from tissue that has not been cleanly severed, or is too large in diameter to fit through the opening in the cutting edge. The clogged cutting instrument must be removed from the arthroscopic site and the suction has to be stopped. Then, a physician or assistant has to manually pull the tissue out of the cutting instrument. Many times, the physician is unable to remove obstructing matter from the instrument and has to use a new instrument to continue surgery. Accordingly, clogging of such cutting instruments can cause a significant time delay in arthroscopic surgery and also result in additional costs due to the use of additional cutting instruments.

Previous inventions have attempted to reduce or eliminate problems associated with clogging of surgical cutting instruments. For example, U.S. Pat. No. 5,782,795 to Bays (Bays) describes a surgical suction cutting instrument with internal irrigation. The apparatus comprises an outer tubular member and an inner tubular member rotatably received in the outer tubular member. The distal end of the inner tubular member forms a cutting edge, and an aspiration lumen is formed within the inner tubular member. Further, an elongate tubular member is attached to the outer tubular member and configured to supply irrigation fluid to the cutting edge of the instrument.

During operation, the rotating cutting edge of the inner member engages and debrides tissue, and irrigation fluid from the elongate tubular member is provided to the cutting chamber. The debrided tissue and irrigation fluid are continuously removed through the aspiration lumen of the inner tubular member. By supplying irrigation fluid to the cutting chamber, the fluid can flush tissue and reduce clogging in the cutting chamber.

The device described in the Bays patent has several drawbacks. First, flow through the aspiration lumen is never interrupted, and therefore, if tissue becomes clogged in the cutting chamber, the aspiration may complicate the clogging despite the provision of irrigation fluid. Further, the elongate tubular member attached to the outer tubular member adds to the profile of the device and is not easily adapted for use with existing surgical suction cutting instruments. This is because the elongate tubular member must be attached to the outer tubular member and then placed in fluid communication with the cutting chamber, making for a difficult retrofit into existing instrument designs.

In view of these drawbacks of previously known systems, it would be desirable to provide apparatus and methods for rapidly and efficiently removing obstructing matter from a surgical cutting instrument.

It further would be desirable to provide apparatus and methods for removing obstructing matter from a surgical cutting instrument without removing it from the surgical site, thereby saving operating time.

It also would be desirable to provide apparatus and methods for removing obstructing matter from a surgical cutting instrument within the surgical site without having to turn off a suction device coupled to the cutting instrument.

It still further would be desirable to provide apparatus and methods for removing obstructing matter from a surgical cutting instrument that can be used in conjunction with existing surgical suction cutting instruments.

### SUMMARY OF THE INVENTION

In view of the foregoing, it is an object of the present invention to provide apparatus and methods for rapidly and efficiently removing obstructing matter from a surgical cutting instrument.

It is also an object of the present invention to provide apparatus and methods for removing obstructing matter from a surgical cutting instrument without removing it from the surgical site, thereby saving operating time.

It is a further object of the present invention to provide apparatus and methods for removing obstructing matter from a surgical cutting instrument within the surgical site without having to turn off a suction device coupled to the cutting instrument.

It is still a further object of the present invention to provide apparatus and methods for removing obstructing matter from a surgical cutting instrument that can be used in conjunction with existing surgical suction cutting instruments.

These and other objects of the present invention are accomplished by providing apparatus comprising means for interrupting and means for flushing. The means for interrupting is configured to interrupt aspiration flow in tubing coupled to the cutting instrument, while the means for flushing is configured to flush fluid in the tubing, situated distal to the means for interrupting, in a distal direction. The means for flushing urges fluid distally, towards the surgical site, to flush obstructions away from the cutting instrument.

In a first embodiment of the present invention, the apparatus comprises an actuation means having a preferably bulb-shaped exterior surface. The actuation means is configured to be used in conjunction with aspiration tubing coupled to the cutting instrument, such that the actuation means surroundingly encloses the aspiration tubing during use.

In this embodiment, the means for interrupting comprises at least one inward protrusion disposed between the bulb-shaped exterior surface and a section of the aspiration tubing. Further, the means for flushing comprises at least one fluid chamber disposed between the exterior surface and the aspiration tubing at a location distal to the means for interrupting.

In operation, the aspiration tubing is configured to aspirate particulate matter, such as debrided tissue and irrigation fluid, from a surgical site. When no external compressive forces are applied to the actuation means, the inward protrusion does not impose substantial forces upon the aspiration tubing, thereby allowing aspiration to be achieved throughout the tubing.

Once a physician determines that an obstruction is present in the cutting instrument, the physician compresses the bulb-shaped exterior of the actuation means to cause the inward protrusion to compress a section of aspiration tubing. This inhibits aspiration in the tubing distal to the inward protrusion.

As the physician further compresses the bulb-shaped exterior, the fluid chamber of the means for flushing is compressed. This causes compression of a section of tubing distal to the means for interrupting. Fluid in the aspiration tubing, distal to the means for interrupting, then is flushed in a distal direction. By causing fluid in the aspiration tubing to flow in a distal direction, the fluid flushes clogged tissue away from the cutting instrument.

Advantageously, in accordance with principles of the present invention, the physician need not remove the cutting instrument from the surgical site or provide a replacement instrument because the obstructing tissue is effectively removed at the surgical site. Further, the physician need not stop the suction device to remove the obstruction, since aspiration is blocked by the means for interrupting. Advantageously, considerable time and money may be saved by not substantially delaying the surgical procedure or using replacement equipment.

In an alternative embodiment of the present invention, the actuation means comprises first and second handles that are configured to actuate first and second rollers. The first and second rollers are configured to move within respective channels in the handles, and serve both as the means for interrupting aspiration and the means for flushing fluid in the aspiration tubing.

In a fully aspirating state, the rollers are disposed at a proximal section of their respective channels, and do not impose substantial forces upon the aspiration tubing. When a physician detects obstructing tissue in the cutting edge of the cutting instrument, the physician may compress the handles to advance the rollers distally within their respective channels. As compression is applied to the handles, the rollers first interrupt aspiration flow in the tubing. Then, the rollers are urged distally to advance fluid in the tubing, distal to the rollers, in a distal direction. The distally urged fluid flushes the surgical site and facilitates removal of clogged tissue from the cutting instrument.

A further alternative embodiment of the present invention employs an actuation means having a preferably bulb-shaped exterior surface and at least one arcuate spring disposed within the exterior surface. A roller is coupled to a distal end of each arcuate spring, and each of the rollers are configured to be advanced within roller guides disposed within the actuation means.

In operation, a physician compresses the bulb-shaped exterior to compress a central region of the arcuate spring. As the spring is compressed, the roller guides urge the rollers in an inward direction to compress the aspiration tubing and interrupt aspiration flow.

As the bulb-shaped exterior of the actuation means is further compressed, the rollers are guided in a distal direction while pinching flow in the tubing. This flushes fluid in the aspiration tubing, situated distal to the rollers, in a distal direction to facilitate removal of obstructing tissue.

In further alternative embodiments of the present invention, the means for interrupting and means for flushing are disposed within a handle of the surgical cutting instrument itself. A physician may actuate these components electronically, e.g., by pressing a button disposed on the handle, or alternatively may actuate the components using mechanical means.

Still further embodiments of the present invention are disclosed. Each embodiment is based on the principle of first interrupting aspiration in aspiration tubing using a means for interrupting, and then flushing fluid in the aspiration tubing, situated distal to the means for interrupting, in a distal direction to flush away clogged particulate matter. Alternative embodiments of the present invention based on these principles are described in detail hereinbelow.

### BRIEF DESCRIPTION OF THE DRAWINGS

Further features of the invention, its nature and various advantages will be more apparent from the accompanying drawings and the following detailed description of the preferred embodiments, in which:

FIG. 1 is a schematic of a hypothetical surgical cutting instrument that may be used in conjunction with apparatus of the present invention;

FIGS. 2A-2B are, respectively, a side view and a side sectional view of a first embodiment of the present invention, which may be used to facilitate removal of obstructions from the cutting instrument of FIG. 1;

FIG. 3A-3C are, respectively, side sectional views of the apparatus of FIGS. 2A-2B in fluid aspirating, interrupting and flushing states;

FIGS. 4A-4C are, respectively, side sectional views of an alternative embodiment of the apparatus of FIGS. 2-3 in fluid aspirating, interrupting and flushing states;

FIGS. 5A-5D are side views of a further alternative embodiment of the present invention in an aspirating state, fluid interrupting states, and a flushing state;

FIGS. 6A-6C are, respectively, side sectional views of an alternative embodiment of the present invention in fluid aspirating, interrupting and flushing states;

FIGS. 7A-7C are, respectively, a side view of a further alternative embodiment of the present invention in a fluid aspirating state, and side sectional views of the apparatus in fluid interrupting and flushing states;

FIG. 8 is an alternative embodiment of the invention described in FIGS. 7A-7C;

FIG. 9 is an alternative embodiment of the present invention showing means for interrupting and means for flushing disposed within a handle of a surgical cutting instrument; and

FIG. 10 is an alternative embodiment of the apparatus of FIG. 9.

### DETAILED DESCRIPTION

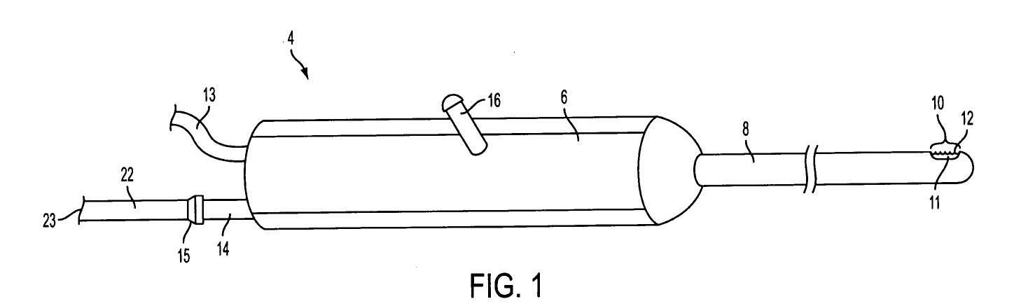

Referring now to FIG. 1, a surgical suction cutting instrument that may be used in conjunction with apparatus of the present invention is described. Surgical suction cutting instrument 4 may be any conventional cutting instrument known in the art, and the particular features depicted in FIG. 1 are provided merely for illustrative purposes. In the embodiment depicted in FIG. 1, cutting instrument 4 comprises handle 6 having proximal and distal ends, and further comprises outer shaft 8 having proximal and distal ends. The proximal end of outer shaft 8 is coupled to the distal end of handle 6, while the distal end of outer shaft 8 comprises opening 10, as depicted in FIG. 1.

Outer shaft 8 houses inner shaft 11 having proximal and distal ends. The proximal end of inner shaft 11 is coupled to a motor (not shown), which is disposed in handle 6 and configured to drive rotation of cutting blade 12 on the distal end of shaft 11 when actuation means 16 is actuated. As will be apparent to those skilled in the art, opening 10 is configured to permit tissue to be cut by rotation of cutting edge 12 with respect to outer shaft 8.

Referring still to FIG. 1, surgical suction cutting instrument 4 further comprises electrical supply means 13 and aspiration line 14 coupled to handle 6. Electrical supply means 13 is coupled to the motor, thereby driving rotation of cutting edge 12.

In FIG. 1, aspiration line 14 is coupled to aspiration tubing 22, which has proximal and distal ends. The proximal end of aspiration tubing 22 is coupled to a suction device (not shown), while the distal end of aspiration tubing 22 is coupled to aspiration line 14 via port 15. Alternatively, port 15 may be omitted, such that aspiration tubing 22 and aspiration line 14 are in effect the same line.

When the suction device is turned on, suction is provided through lumen 23 of aspiration tubing 22, which is in fluid communication with the distal end of outer shaft 8. Accordingly, suction is provided to cutting edge 12 and opening 10 to draw tissue towards the cutting edge, and also to facilitate removal of debrided tissue and irrigation fluid from a surgical site.

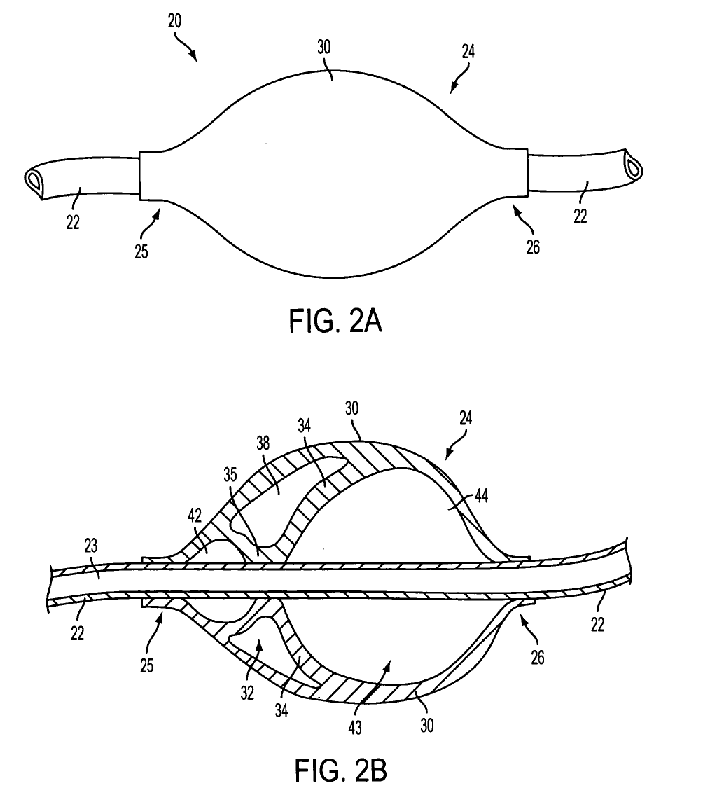

Referring now to FIGS. 2A-2B, a first embodiment of the present invention is described. Apparatus 20 is configured to facilitate removal of clogged tissue from a surgical cutting instrument, such as cutting instrument 4 of FIG. 1.

Apparatus 20 comprises actuation means 24 having proximal and distal regions 25 and 26, respectively, as shown in FIG. 2A. Actuation means 24 is configured to be used in conjunction with at least one piece of aspiration tubing 22 that is in fluid communication with cutting instrument 4, for example, as depicted in FIG. 1.

Actuation means 24 comprises exterior surface 30, which preferably is a bulb-shaped member that is adapted to be grasped by a human hand. Exterior surface 30 may be manufactured using a suitable compound, such as rubber, that allows the exterior surface to be compressed in an inward direction when a force is applied, and then return to its original, non-compressed state when the compressive force is removed.

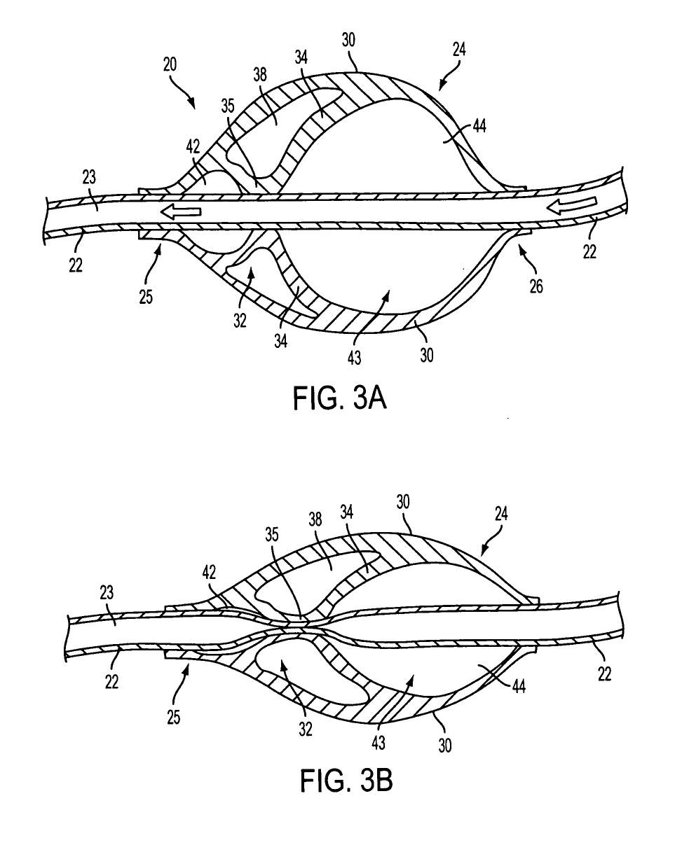

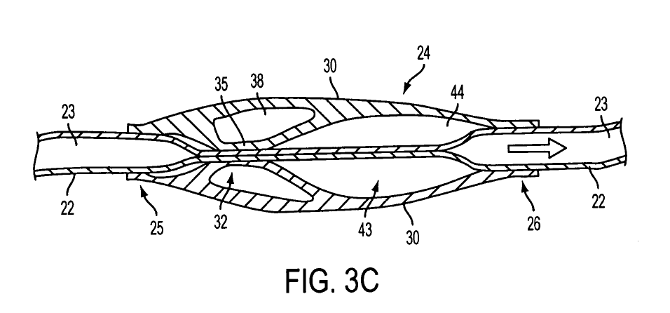

Apparatus 20 further comprises means for interrupting 32, as shown in FIG. 2B. In the embodiment of FIGS. 2-3, means for interrupting 32 comprises at least one inward protrusion 34 that is disposed within exterior surface 30 and configured to selectively compress aspiration tubing 22. Inward protrusion 34 preferably has a curved shape with at least one apex or engagement point 35, as shown in FIG. 2B. Apex 35 of inward protrusion 34 is configured to selectively compress an exterior surface of aspiration tubing 22, and may fully encircle tubing 22, contact the tubing at one location, contact the tubing at two opposing locations, or contact the tubing in any other manner suitable for applying compressive force upon tubing 22.

If apex 35 fully encircles tubing 22, then the provision of a bulb-shaped exterior surface 30 may be desirable. However, where two opposing apexes are provided, it may be desirable to provide exterior surface 30 with indicia (not shown) corresponding to the circumferential positioning of apexes 35 beneath exterior surface 30. Such indicia allows a user to compress exterior surface 30 at a location that will most effectively cause compression of the apexes to actuate the device, as described hereinbelow. Alternatively, when two opposing apexes 35 are employed, exterior surface 30 may comprise an elliptical or oval shape, so that a physician can simply compress the opposing surfaces to effectively cause compression of the apexes.

Inward protrusion 34 may be formed from the same material as exterior surface 30 of actuation means 24, or alternatively, may be manufactured using a separate material that is bonded to an interior region of exterior surface 30. If a separate material is used, it may be desirable to provide inward protrusion 34 as a more rigid member than exterior surface 30, so that compression of exterior surface 30 will result in a stronger and more direct compression of tubing 22.

Apparatus 20 may comprise at least one proximal fluid chamber 42 formed between aspiration tubing 22 and exterior surface 30, at a location proximal to apex 35. Further, recess 38 may be formed between inward protrusion 34 and exterior surface 30, as depicted in FIG. 2B. Alternatively, these regions may be solid, such that recess 38 and/or fluid chamber 42 are omitted entirely.

Referring still to FIG. 2B, apparatus 20 further comprises means for flushing 43, which in the embodiment of FIGS. 2-3 comprises at least one fluid chamber 44 formed between aspiration tubing 22 and exterior surface 30. Fluid chamber 44 of means for flushing 43 is disposed at a location distal to apex 35, as shown in FIG. 2B.

Referring now to FIGS. 3A-3C, use of apparatus 20 of FIGS. 2A-2B is described for facilitating removal of clogged tissue from cutting instrument 4 during a surgical procedure, such as arthroscopic surgery. As will be apparent to one skilled in the art, irrigating fluid may be delivered to the surgical site, while aspiration tubing 22 is employed to aspirate irrigating fluid and cut tissue from the surgical site. An irrigation line (not shown) may be used in conjunction with apparatus 20 of the present invention, if desired.

In FIG. 3A, actuation means 24 is shown in an “open” or fully aspirating state. The proximal end of aspiration tubing 22 is coupled to a suction device, and aspiration tubing 22 is in fluid communication with the distal end of cutting instrument 4. Accordingly, irrigation fluid and cut tissue from the surgical site will be aspirated through opening 10 of cutting instrument 4, and then through tubing 22 in a proximal direction, as indicated by the arrows in FIG. 3A.

As noted hereinabove, one common problem associated with use of conventional surgical cutting instruments during arthroscopic procedures is the tendency of cut tissue to clog opening 10 or cutting edge 12 of the cutting instrument. In accordance with one aspect of the present invention, once the cutting instrument becomes clogged, a physician may manually compress exterior surface 30 to cause apex 35 to compress a section of aspiration tubing 22, as shown in FIG. 3B. This inhibits aspiration distal to apex 35, such that no substantial fluid flow occurs in tubing 22 distal to apex 35.

Referring now to FIG. 3C, when a physician applies further compression to actuation means 24, means for flushing 43 is actuated. Specifically, the compression of exterior surface 30 towards tubing 22 causes fluid in chamber 44 to compress tubing 22. The compression of tubing 22 causes irrigation fluid in tubing 22 that is distal to apex 35 to be urged in a distal direction, i.e., towards opening 10 and cutting edge 12. Since apex 35 remains sealingly engaged with aspiration tubing 22, fluid in tubing 22 will be flushed in a distal direction.

By applying pressure to fluid in tubing 22 and flushing fluid in a distal direction, the distally flowing fluid flushes clogged tissue away from opening 10 and cutting edge 12 of the cutting instrument. Once compression is released, this tissue then may be introduced back into the cutting instrument, reduced in size, and then effectively aspirated through tubing 22.

Advantageously, in accordance with one aspect of the present invention, a physician may remove obstructing tissue from the surgical cutting instrument using actuation means 24 without having to remove the cutting instrument from the surgical site and manually remove the clogged tissue. Also, the surgeon is not expected to have to replace the cutting instrument with a different instrument, thereby saving time and money.

Upon successful removal of clogged tissue, the physician can remove the compressive forces imposed upon actuation means 24, thereby causing actuation means 24 to return to its original shape, depicted in FIG. 3A. At this time, aspiration throughout the tubing is restored.

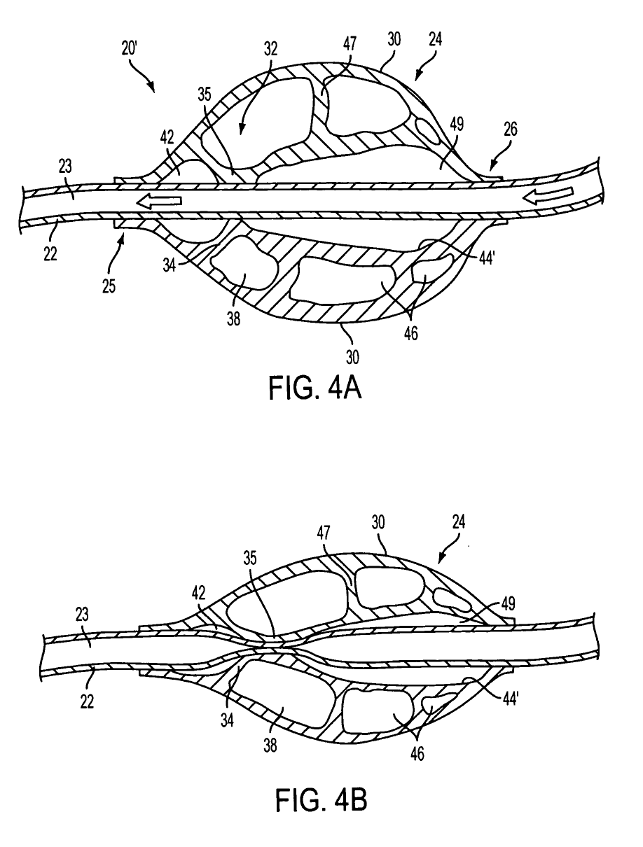

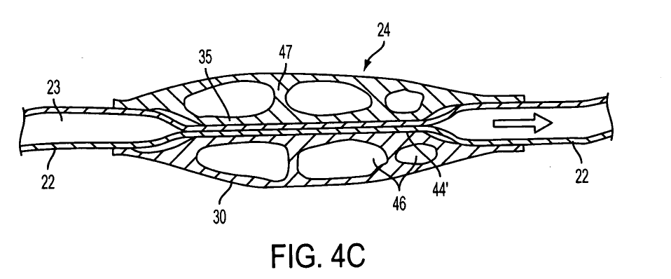

Referring now to FIGS. 4A-4C, an alternative embodiment of apparatus 20 of FIGS. 2-3 is described. Apparatus 20′ is similar to apparatus 20, except as noted hereinbelow. In particular, an alternative means for flushing, comprising at least one interior compression member 44′, is employed. Interior compression member 44′ is configured to apply a direct compressive force to aspiration tubing 22, as described hereinbelow.

Apparatus 20′ preferably further comprises at least one support structure 47 disposed between exterior surface 30 and interior compression member 44′, as shown in FIG. 4A. Fluid chambers 46 may be formed between support structures 47, exterior surface 30, and/or interior compression member 44′. Support structures 47 help translate compressive forces from exterior surface 30 to interior compression member 44′, while fluid chambers 46 conform to provide flexibility and comfort during operation.

The operation of apparatus 20′ is similar to use of apparatus 20, as described in FIGS. 3A-3C hereinabove. In a first step, actuation means 24 is provided in an “open” or fully aspirating state, as shown in FIG. 4A. Once cutting instrument 4 becomes clogged, a physician may manually compress exterior surface 30 of actuation means 24 to cause apex 35 of means for interrupting 34 to sealingly compress a section of aspiration tubing 22, as shown in FIG. 4B. This inhibits aspiration distal to apex 35.

Referring now to FIG. 4C, apparatus 20′ is shown when a physician applies further compression to exterior surface 30 of actuation means 24. The compression of exterior surface 30 towards tubing 22 causes support structures 47 to translate the compressive force to interior compression member 44′, which in turn directly compresses a portion of tubing 22 distal to apex 35. The direct compression of tubing 22 causes irrigation fluid in tubing 22 that is distal to apex 35 to be urged in a distal direction, i.e., towards opening 10 and cutting edge 12. As described hereinabove, by applying pressure to fluid in tubing 22 and causing the fluid to flow in a distal direction, the fluid flushes clogged tissue away from the cutting edge of the instrument.

In the embodiments of FIGS. 2-4 hereinabove, it will be apparent to one skilled in the art that either air or liquid may be disposed within chambers 38, 42, 44, 46 and 49. In particular, the provision of liquid in chambers 44, 46 and 49 is expected to facilitate compression of tubing 22. Alternatively, any of the fluid chamber depicted hereinabove may be omitted and replaced with solid regions.

In an alternative embodiment, fluid trapped in chambers 44 of FIGS. 3A-3C may be delivered to aspiration tubing 22 to facilitate removal of clogged tissue in the cutting instrument. In this embodiment, a section of tubing 22, situated between apex 35 and distal region 26, may comprise a plurality of small perforations (not shown). In the step described in FIG. 3C, fluid disposed in chamber 44 may be infused into tubing 22 via the plurality of small perforations. The infused fluid then will flow in a distal direction through lumen 23 to facilitate removal of clogged tissue. Similarly, for the embodiment described in FIGS. 4A-4C, perforations may be provided in tubing 22 and liquid disposed in chambers 49 may be infused into lumen 23 upon compression of exterior surface 30.

Further, as will be apparent to one skilled in the art, varying degrees of aspiration tubing stiffness may be provided. For example, relatively flexible aspiration tubing 22 may be provided when air is disposed in chambers 44 and 49, to ensure that the air may compress the tubing. By contrast, relatively rigid aspiration tubing may be provided when liquid is disposed in chambers 44 and 49 to facilitate compression of the relatively stiff tubing. In any embodiment, tubing 22 also may comprise at least one relatively flexible segment and at least one relatively rigid segment.

In accordance with another aspect of the present invention, it should be noted that apparatus 20 may be used in conjunction with any existing cutting instrument 4. Apparatus 20 may be provided securely disposed about tubing 22, or alternatively, apparatus 20 may be provided as a separate component. In the latter case, apparatus 20 may slide over tubing 22 and a user may secure proximal and distal regions 25 and 26 to tubing 22 at a desired location on the tubing. For example, apparatus 20 may be secured about tubing 22 using a suitable adhesive, thermal plastic bond, or using mechanical means such as clamps. A physician therefore may vary the longitudinal positioning of apparatus 20 with respect to tubing 22. However, it may be desirable to have apparatus 20 disposed towards the distal end of tubing 22 so that the apparatus is in relatively close proximity to cutting instrument 4.

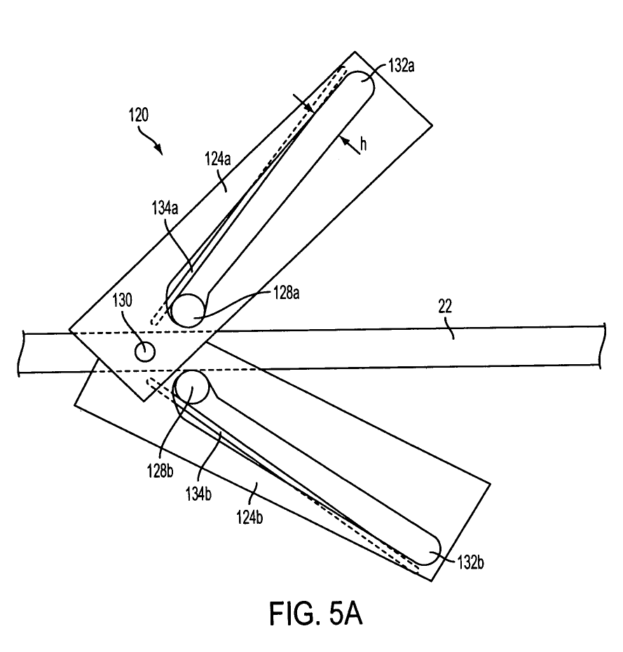

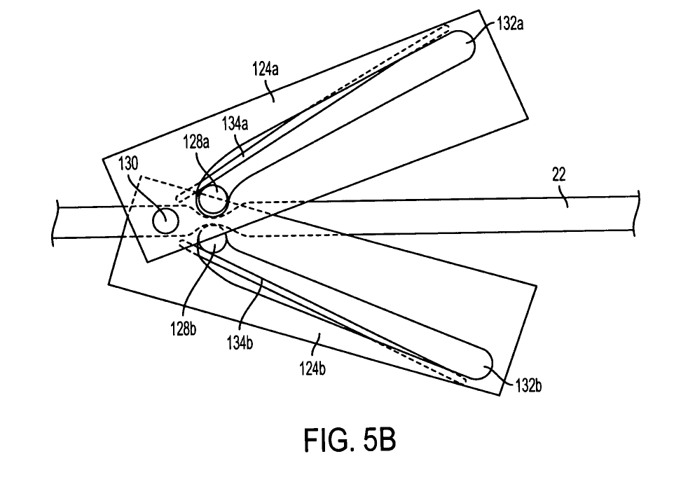

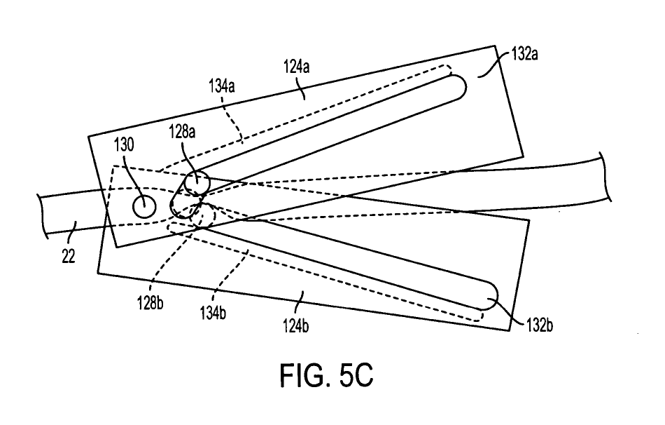

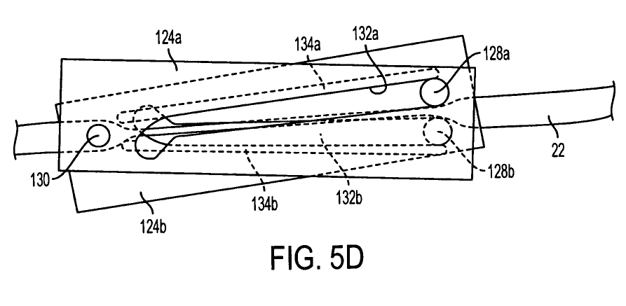

Referring now to FIGS. 5A-5D, an alternative embodiment of the present invention is described for facilitating removal of clogged tissue during a surgical procedure. Apparatus 120 comprises an actuation means having first handle 124a and second handle 124b. First handle 124a has channel 132a disposed therein, while second handle 124b has channel 132b disposed therein, as shown in FIG. 5A. First and second handles 124a and 124b are coupled together at their respective proximal ends using pivot pin 130, which is disposed to partially or fully surround aspiration tubing 22.

Apparatus 120 preferably further comprises springs 134a and 134b, each having proximal and distal ends. The proximal ends of springs 134a and 134b preferably are coupled to the proximal ends of handles 124a and 124b, respectively, while the distal ends of springs 134a and 134b are coupled to the distal ends of handles 124a and 124b, respectively, as shown in FIG. 5A. Alternatively, the proximal ends of the springs may be left unsecured, so that the proximal ends of the springs may deflect when the device is actuated, as described further in FIGS. 5C-5D hereinbelow.

Apparatus 120 further comprises means for interrupting and means for flushing. In the embodiment depicted herein, both the means for interrupting and means for flushing are the same, and they comprise first and second rollers 128a and 128b. First and second rollers 128a and 128b preferably comprise an outer diameter that is slightly smaller than height h of channels 132a and 132b, thereby permitting the rollers to move longitudinally within their respective channels, as described hereinbelow.

Referring still to FIG. 5A, apparatus 120 is depicted in an “open” or fully aspirating state, whereby the distal ends of handles 124a and 124b are widely separated. In the aspirating state, rollers 128a and 128b are disposed in proximal sections of their respective channels. Rollers 128a and 128b do not apply substantial forces to tubing 22 in the fully aspirating state.

Referring now to FIG. 5B, a physician may realize cutting instrument 4 has become clogged during an surgical procedure. When this occurs, the physician applies a compressive force to first and second handles 124a and 124b. The compressive force causes the proximal ends of the handles to rotate about pivot point 130, and further causes the distal ends of the handles to be drawn closer together, as depicted in FIG. 5B.

As handles 124a and 124b are compressed together, rollers 128a and 128b apply a compressive force upon aspiration tubing 22, as shown in FIG. 5B. The compressive force of the rollers pinches the tubing and inhibits further aspiration of fluid in lumen 23 (distal to the rollers). When relatively light compressive forces are applied to handles 124a and 124b, springs 134a and 134b do not substantially displace, and therefore serve to confine rollers 128a and 128b at the proximal sections of their respective channels.

Referring now to FIG. 5C, as a physician further compresses handles 124a and 124b, proximal regions of springs 134a and 134b are displaced against the rollers. Displacement of springs 134a and 134b, above a predetermined compression threshold, causes rollers 128a and 128b to advance distally within their respective channels.

Referring now to FIG. 5D, still further compression of handles 124a and 124b causes rollers 128a and 128b to be advanced towards the distal end of channels 132a and 132b, respectively. Rollers 128a and 128b remain engaged with tubing 22, such that their advancement flushes fluid in lumen 23, situated distal to the rollers, in a distal direction.

By applying pressure to fluid in tubing 22 and flushing the fluid in a distal direction, the fluid flow flushes clogged tissue from opening 10 and cutting edge 12. Upon successful removal of clogged tissue, the physician can remove the previously-applied compressive forces imposed upon handles 124a and 124b, thereby causing the handles to return to their original positions, depicted in FIG. 5A.

As will be apparent to one skilled in the art, characteristics of springs 134a and 134b may be varied to vary the manual force required to actuate apparatus 120. However, the springs preferably are stiff enough so that they do not substantially displace until tubing 22 first is compressed. After a threshold force is applied to compress tubing 22, then the springs are configured to displace enough to permit rollers 128a and 128b to be advanced distally in channels 132a and 132b.

Further, the distance between pivot point 130 and the proximal ends of channels 132a and 132b may be varied to vary the force required to actuate apparatus 120. For example, increasing this distance may reduce the force required to actuate apparatus 120, since greater leverage is provided.

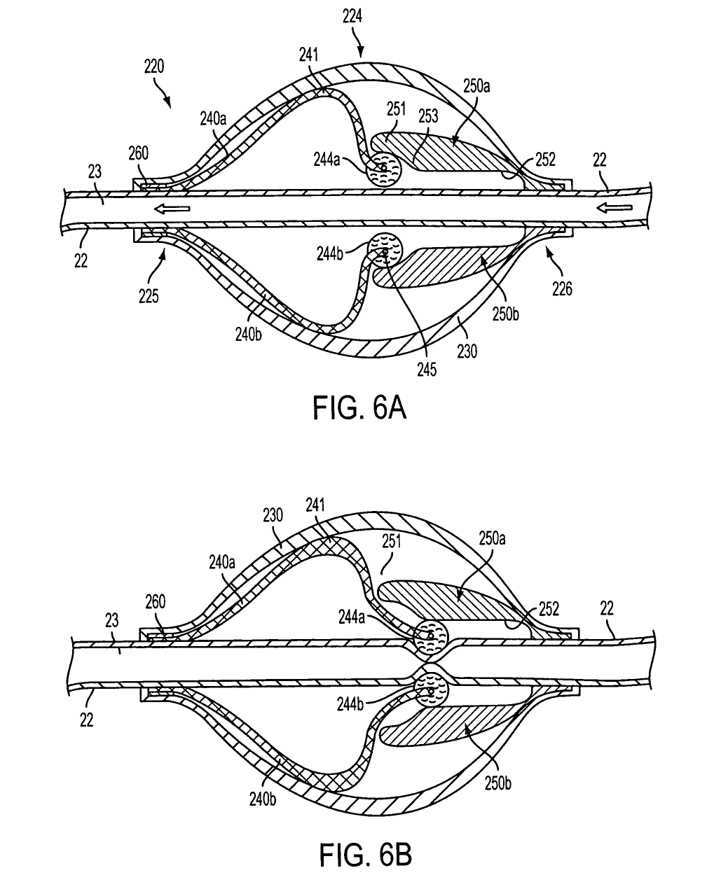

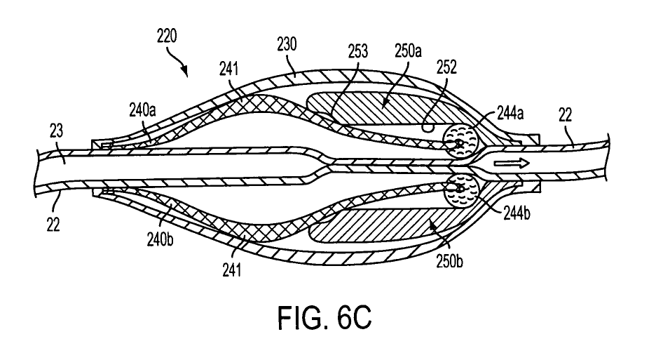

Referring now to FIGS. 6A-6C, a further alternative embodiment of the present invention is described for clearing obstructions from cutting instrument 4. Apparatus 220 comprises actuation means 224, which comprises exterior surface 230. Apparatus 220 further comprises means for interrupting and means for flushing, which, in the embodiment of FIGS. 6A-6C, are the same. The means for interrupting and the means for flushing comprise first and second arcuate springs 240a and 240b, and further comprise rollers 244a and 244b.

Arcuate springs 240a and 240b have proximal and distal ends and a central region 241 disposed therebetween. The proximal ends of arcuate springs 240a and 240b are fixedly attached to an interior portion of exterior surface 230 at fixation point 260, as shown in FIG. 6A. The distal ends of arcuate springs 240a and 240b are coupled to rollers 244a and 244b, respectively. Rollers 244a and 244b preferably are coupled to their respective springs using a central pin 245, as depicted in FIG. 6A.

When no external forces are applied to apparatus 220, central regions 241 of arcuate springs 240a and 240b are configured to assume the curved configuration depicted in FIG. 6A. When compressive forces are applied, the arcuate springs may be deformed accordingly, as will be described in greater detail hereinbelow.

Apparatus 220 further comprises first and second roller guides 250a and 250b, each having proximal region 251 and distal region 252. Each proximal region 251 transitions into distal region 252 via curvature or slant 253. Each proximal region 251 is configured to house rollers 244a and 244b in the aspirating state, as depicted in FIG. 6A and described in greater detail hereinbelow.

In accordance with one aspect of the present invention, apparatus 220 is configured for use with existing surgical cutting instruments, such as cutting instrument 4 of FIG. 1. Apparatus 220 may be affixed to tubing 22, for example, at proximal and distal regions 225 and 226, or alternatively, apparatus 220 may slide longitudinally over the tubing. In the latter embodiment, a physician may insert the tubing through a central region of apparatus 220, and then position apparatus 220 at a desired location with respect to the tubing.

The operation of apparatus 220 is similar to use of apparatus 20, as described in FIGS. 3A-3C hereinabove. In a first step, apparatus 220 is provided in an “open” or fully aspirating state, whereby no substantial external forces are applied to apparatus 220. In this state, central regions 241 of arcuate springs 240a and 240b assume the curved configuration depicted in FIG. 6A. At this time, rollers 244a and 244b are disposed within proximal regions 251 of their respective roller guides 250a and 250b. Accordingly, rollers do not impose a substantial force upon tubing 22 during the aspirating state, as shown in FIG. 6A. As noted above, when the proximal end of tubing 22 is coupled to a suction device (not shown), irrigation fluid and cut tissue from the surgical site will be aspirated through aspiration tubing 22 in a proximal direction, as indicated by the arrows in FIG. 6A.

Referring now to FIG. 6B, once suction tubing 22 becomes clogged, a physician may manually compress exterior surface 230 to compress central region 241 of arcuate springs 240a and 240b. Compression of the arcuate springs causes rollers 244a and 244b to advance distally, since the proximal ends of the arcuate springs remain fixed at points 260.

When compression is applied, slant 253 urges rollers 244a and 244b in an inward direction, i.e., towards tubing 22, as shown in FIG. 6B. Rollers 244a and 244b sealingly compress a section of aspiration tubing 22, thereby inhibiting suction within lumen 23 (distal to the rollers).

Referring now to FIG. 6C, apparatus 220 is shown when a physician applies further compression to exterior surface 230 of actuation means 224. The compression of exterior surface 230 towards aspiration tubing 22 urges springs 240a and 240b to straighten, thereby advancing rollers 244a and 244b in a distal direction, as shown in FIG. 6C. The rollers are guided by distal region 252 of roller guides 250a and 250b, which ensure that the rollers remain firmly engaged with tubing 22. The advancement of rollers 244a and 244b causes irrigation fluid in tubing 22 that is distal to the rollers to be flushed in a distal direction, i.e., towards opening 10 and cutting edge 12, thereby flushing obstructions away from the cutting instrument.

Upon successful removal of clogged tissue, the surgeon can remove the previously-applied compressive forces, thereby causing springs 240a and 240b to return to their preferred arcuate shapes, as depicted in FIG. 6A. At this time, aspiration throughout the tubing is restored, and will remain in the aspirating state until the surgeon applies further compressive forces to remove subsequently clogging tissue.

As will be apparent to one skilled in the art, characteristics of springs 240a and 240b may be varied to vary the manual force required to actuate apparatus 220. However, the springs should be stiff enough to cause rollers 244a and 244b to compress tubing 22 without substantially bowing inward first, i.e., the springs will not merely bow inward before the tubing is displaced. After a threshold force is applied to compress tubing 22, then the springs are configured to displace enough to permit rollers 244a and 244b to be advanced distally in channels 250a and 250b.

Further, it will be apparent to one skilled in the art that, in lieu of two or more roller guides 250a and 250b, one continuous, circumferentially-shaped roller guide 250 may be disposed within exterior surface 230. Such a continuous and circumferential guide may be adapted to guide each roller 244. In such an embodiment, a bulb-shaped exterior surface 230 may be desirable.

However, where two rollers and two roller guides are employed, as depicted in FIGS. 6A-6C, it may be desirable to provide exterior surface 230 with indicia (not shown) corresponding to the circumferential positioning of springs 244a and 244b beneath exterior surface 230. Such indicia allows a user to compress exterior surface 230 at a location that will most effectively actuate springs 240a and 240b. Alternatively, when two springs and two roller guides are employed, exterior surface may comprise an elliptical or oval shape, so that a physician can simply compress the opposing regions to effectively actuate the device.

Finally, it will be apparent that although two rollers 244a and 244b are depicted, greater or fewer rollers may be employed to achieve the effects described hereinabove.

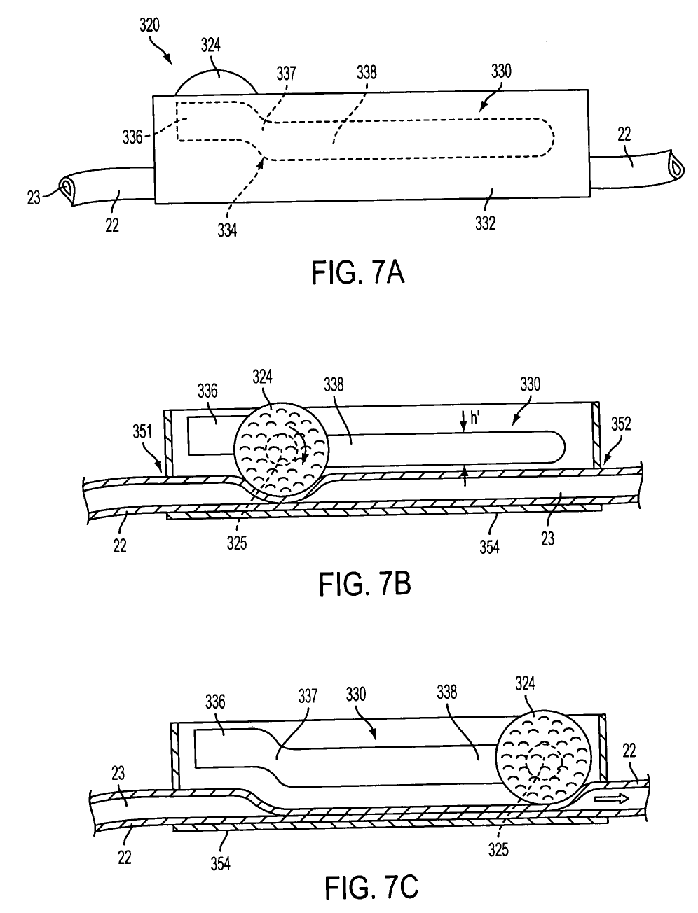

Referring now to FIGS. 7A-7C, a further alternative embodiment of the present invention is described. Apparatus 320 comprises roller 324 and housing 330, which are configured to be used in conjunction with aspiration tubing 22 having lumen 23 disposed therethrough. In this embodiment, roller 324 serves as the actuation means, and also as the means for interrupting and means for flushing, as will be described in greater detail hereinbelow.

Housing 330 preferably comprises a rectangular shape, although other shapes may be employed. Channel 334 having proximal region 336, central region 337 and distal region 338 is disposed within housing 330, as shown in FIG. 7A. In a preferred embodiment, distal region 338 spans a greater length than proximal region 336.

Roller 324 is coupled to roller axle 325, as shown in FIG. 7B. Roller axle 325 preferably has an outer diameter that is slightly smaller than height h′ of channel 334, thereby permitting roller 324 to be advanced longitudinally within channel 334 via roller axle 325.

Tubing 22 is disposed through proximal and distal openings 351 and 352 of housing 330, as depicted in FIG. 7B. The tubing may be inserted through the proximal and distal openings when roller 324 is situated in proximal region 336 of channel 334, as shown in FIG. 7A. Proximal and distal openings 351 and 352 preferably are disposed just above lower surface 354 of housing 330, so that tubing 22 can rest upon lower surface 354 during operation.

A physician may advance apparatus 320 longitudinally with respect to tubing 22 until housing 330 is disposed in a desired location with respect to the tubing. The physician then may secure the position of housing 330 with respect to tubing 22, e.g., using an adhesive or mechanical means, or leave the housing unsecured.

Referring to FIG. 7A, apparatus 220 is provided in an “open” or fully aspirating state, whereby roller 324 is disposed in proximal region 336 of channel 334. At this time, roller 324 is constrained at such a distance from tubing 22 that the roller does not impose a substantial compressive force upon the tubing. This permits aspiration through tubing 22 when the proximal end of the tubing is coupled to a suction device.

Referring now to FIG. 7B, once a cutting instrument coupled to tubing 22 becomes clogged during a surgical procedure, a physician may advance roller 324 distally, either manually or using mechanical means (not shown). It should be noted that roller 324 may be round, as depicted, or alternatively may comprise one or more grooves or raised surfaces to provide a frictional resistance that facilitates actuation by the physician.

As roller 324 is advanced distally, the contours of central region 337 cause roller axle 325 to direct roller 324 in an inward direction, i.e., towards tubing 22. Roller 324 subsequently pinches off a section of tubing 22 by compressing the tubing between the roller and lower surface 354 of housing 330, as shown in FIG. 7B. This advancement of roller 324 inhibits suction within lumen 23 (distal to the roller).

Referring now to FIG. 7C, apparatus 320 is shown when a physician further advances roller 324 distally. Roller axle 325 is guided along distal region 338 of channel 334. During this time, roller 324 applies continuous compression upon tubing 22, which is compressed against lower surface 354.

The advancement of roller 324 causes irrigation fluid in tubing 22 that is distal to roller 324 to be flushed in a distal direction, i.e., towards opening 10 and cutting edge 12. As described hereinabove, by applying pressure to fluid in tubing 22 and causing the fluid to flow in a distal direction, the fluid flushes clogged tissue away from cutting edge 12.

Upon successful removal of clogged tissue, a surgeon can retract roller 324 proximally into proximal region 336, thereby causing roller 324 to become fully or substantially disengaged from tubing 22. Tubing 22 will return to its uncompressed shape and aspiration will be restored throughout the tubing.

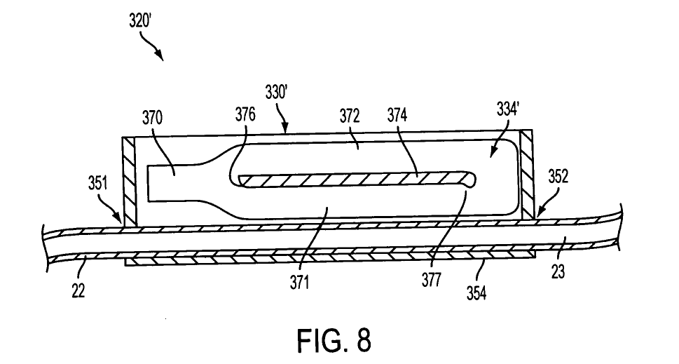

Referring now to FIG. 8, an alternative embodiment of apparatus 320 of FIGS. 7A-7C is described. In FIG. 8, apparatus 320′ functions similarly to apparatus 320, except as noted hereinbelow. Apparatus 320′ comprises housing 330′ having channel 334′. Channel 334′ comprises proximal section 370, advancement channel 371 and return channel 372, which preferably are separated by partition 374. Apparatus 320′ further comprises roller 324 of FIGS. 7A-7C, which is omitted from FIG. 8 for clarity.

In operation, roller 324 is positioned within proximal section 370 of channel 334′ in the aspirating state. Once the cutting instrument becomes clogged, a physician may advance roller 324 distally towards one-way guide 376 of partition 374. One-way guide 376 causes roller 324 to be advanced distally into advancement channel 371.

When roller 324 is advanced into advancement channel 371, roller 324 subsequently pinches off a section of tubing 22 by compressing the tubing between the roller and lower surface 351 of housing 330. At this time, roller 324 inhibits suction within lumen 23 (distal to the roller). As roller 324 is further advanced in advancement channel 371, irrigation fluid in tubing 22 that is distal to the roller is flushed in a distal direction, i.e., towards cutting edge 12.

When roller 324 is advanced toward the distal end of advancement channel 371, distal stop 377 of partition 374 may provide resistance to roller axle 325 (see FIGS. 7B-7C). A surgeon may apply a slightly greater force upon roller 324 to cause the roller axle to be advanced beyond distal stop 377.

Once the roller axle is advanced beyond distal stop 377, the roller is returned to proximal region 370 via return channel 372. In one embodiment, a physician may manually advance roller 324 in a proximal direction through return channel 372 and into proximal region 370. Alternatively, as will be apparent to one skilled in the art, a spring mechanism (not shown) may be employed to facilitate the return of roller 324 to proximal region 370, after the roller passes distal stop 377. It should be noted that, in either the manual or spring-return embodiments, distal stop 377 also may be omitted entirely.

With respect to all embodiments described hereinabove, it will be apparent to one skilled in the art that the means for interrupting and means for flushing may be completely separate entities, each actuated using its own separate actuation means. Specifically, the means for interrupting may compress a first section of tubing 22 to interrupt aspiration within the tubing. Then, the means for flushing, which is disposed at a second location along tubing 22 distal to the means for interrupting, subsequently may be actuated to cause irrigation fluid to be flushed towards cutting edge 12.

Additionally, in other embodiments, the means for flushing may be configured to be pressurized. For example, a desired amount of pressure may be built up in a bulb-shaped compressor. Once a desired pressure is reached, the means for flushing is actuated to permit pressurized fluid to compress tubing 22, thereby resulting in high velocity fluid flow through opening 10 of the cutting instrument. If desired, such pressurized means for flushing can be configured to automatically release fluid once a predetermined pressure threshold is achieved.

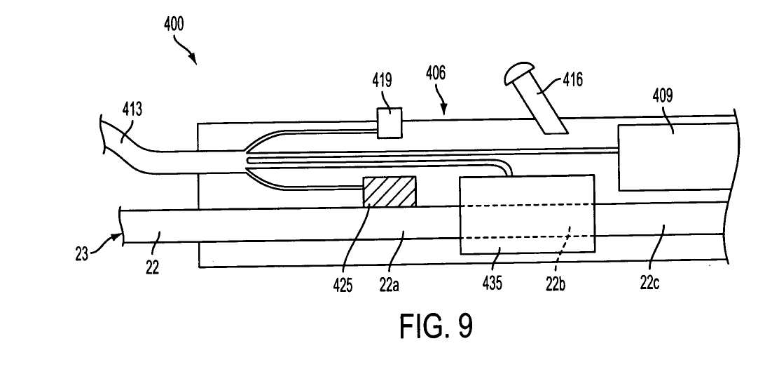

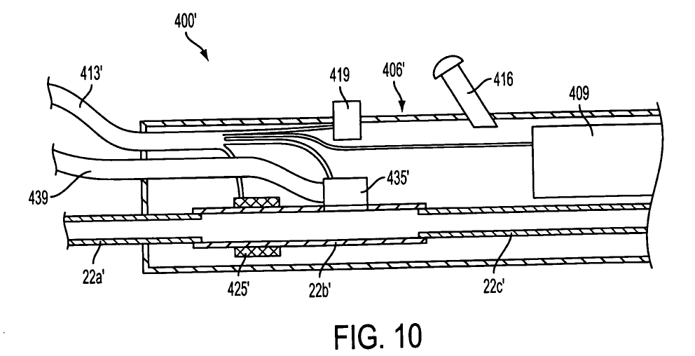

Referring now to FIGS. 9-10, further alternative embodiments of the present invention are described. The embodiments of FIGS. 2-8 described apparatus for facilitating removal of obstructions from a surgical cutting instrument, whereby the apparatus was disposed substantially or exclusively on a section of aspiration tubing external to the cutting instrument. In the embodiments of FIGS. 9-10, similar apparatus are described; however, these embodiments are substantially integrated into a handle of the cutting instrument itself.

Referring to FIG. 9, apparatus 400 comprises cutting instrument handle 406 having proximal and distal ends. Handle 406 is similar to handle 6 of cutting instrument 4 of FIG. 1, except as noted hereinbelow. Like the embodiment of FIG. 1, the distal end of handle 406 of FIG. 9 may be coupled to outer shaft 8 having distal opening 10 (see FIG. 1).

Apparatus 400 further comprises motor 409, which is disposed within handle 406 and configured to drive inner shaft 11 of FIG. 1. Electrical supply means 413 is coupled to handle 406, and is adapted to communicate with multiple components of apparatus 400, as described hereinbelow.

Aspiration tubing 22 having proximal and distal ends also is provided. The proximal end of aspiration tubing 22 is coupled to a suction source (not shown), while the distal end is configured to be selectively placed in fluid communication with cutting edge 12 of the cutting instrument (see FIG. 1).

Apparatus 400 further comprises means for interrupting 425 and means for flushing 435. Means for interrupting 425 is disposed within handle 406 proximal to means for flushing 435, and preferably is disposed adjacent tubing section 22a, as depicted in FIG. 9. Means for interrupting 425 communicates with electrical supply means 413, and further communicates with actuation means 419.

Means for flushing 435 is disposed distal to means for interrupting 425, as depicted in FIG. 9, and also communicates with electrical supply means 413 and actuation means 419. Means for flushing 435 may fully encircle tubing section 22b, as depicted in FIG. 9, or may be disposed adjacent tubing section 22b.

In operation, a proximal end of electrical supply means 413 is coupled to a power source (not shown) and the proximal end of tubing 22 is coupled to a suction source (not shown). The apparatus may be provided in an “open” or fully aspirating state, permitting aspiration of fluid and cut tissue in a proximal direction through tubing 22. In the fully aspirating state, neither means for interrupting 425 nor means for flushing 435 applies substantial compressive forces upon tubing 22.

Once the cutting instrument becomes clogged, a physician may actuate means for interrupting 425, e.g., via actuation means 419. Like the embodiments described hereinabove, means for interrupting 425 is configured to apply compression upon tubing section 22a when actuated, thereby interrupting aspiration flow in tubing 22 (distal to section 22a).

In a next step, means for flushing 435 is actuated, e.g., via actuation means 419. Also like the embodiments described hereinabove, means for flushing 435 is actuated to apply compressive forces upon tubing section 22b, thereby flushing fluid in a distal direction. Distally urged fluid flushes clogged tissue away from cutting edge 12 of the cutting instrument.

Advantageously, in the embodiment of FIG. 9, means for interrupting 425 and means for flushing 435 may comprise various mechanically or electrically actuated means to apply compression to tubing sections 22a and 22b, respectively. For example, means for interrupting 425 may comprise any valve, solenoid, or other mechanism known in the art that may apply a compressive force upon tubing section 22a to compress the tubing in a manner as described hereinabove.

Similarly, means for interrupting 435 may comprise any valve, solenoid or pneumatic pump configured to apply a compressive force upon tubing section 22b. In a particularly useful embodiment, means for flushing 435 is configured to apply a circumferential compressive force upon tubing section 22b in a proximal to distal direction.

As will be apparent to one skilled in the art, a microprocessor and memory (not shown) may be employed to facilitate actuation of means for interrupting 425 and/or means for flushing 435. In response to data instructions received from the microprocessor, means for interrupting 425 and means for flushing 435 may be actuated accordingly.

In a particularly useful embodiment, the microprocessor is programmed to cause means for interrupting 425 to be actuated when actuation means 419, e.g., a button, is pressed once. The microprocessor then automatically instructs means for flushing 435 to be actuated at a later time, e.g., one second later. Such programming is particularly useful because it eliminates the need for a physician to actuate button 419 multiple times to achieve one cycle of flushing.

Alternatively, as will be apparent to one skilled in the art, actuation means 419 may comprise a multi-position switch configured to mechanically actuate means for interrupting 425 and means for flushing 435 in sequence. For example, when actuation means 419 comprises a button, and the button is partially depressed, means for interrupting 425 may be actuated. Then, when the button is fully depressed, means for flushing 435 subsequently is actuated.

Although one actuation means 419 has been described for actuating both means for interrupting 425 and means for flushing 435, other designs may be employed. For example, actuation means 419 may be configured to actuate means for interrupting 425 only, while a second actuation means (not shown) may be disposed on handle 406 to actuate means for flushing 435. In this embodiment, the microprocessor may have instructions to ensure that means for flushing 435 cannot be actuated when means for interrupting 425 is in an open position.

Further, as will be apparent to one skilled in the art, mechanical means similar to those described hereinabove with respect to FIGS. 2-8 may be employed to actuate means for interrupting 425 and/or means for flushing 435. For example, a bladder (not shown) may be disposed within handle 406 adjacent means for interrupting 425 and/or means for flushing 435. In this embodiment, a physician may compress one or more external regions of handle 406, which in turn compresses the bladder adjacent to means for interrupting 425 and/or means for flushing 435. This, in turn, causes compression of a corresponding section of tubing 22.

In still further alternative embodiments of the device described in FIG. 9, means for interrupting 425 and/or means for flushing 435 may comprise one or more rollers that are configured to compress tubing 22, as described generally hereinabove with respect to FIGS. 5-8. In this embodiment, when actuation means 419 is actuated, a microprocessor (not shown) may provide instructions to one or more components, such as linkages, to cause the roller or rollers to compress tubing 22 as desired locations.

In yet a further alternative embodiment, actuation of means for interrupting 425 and means for flushing 435 may be achieved using a foot pedal (not shown). In this embodiment, the foot pedal may be coupled to a microprocessor that is programmed to actuate means for interrupting 425 and/or means for flushing 435, for example, when the foot pedal is depressed one or more times.

Referring now to FIG. 10, a further alternative embodiment of the present invention is described. In FIG. 10, apparatus 400′ is similar to apparatus 400 of FIG. 9, with pertinent exceptions noted hereinbelow. Apparatus 400′ comprises irrigation supply line 439 having proximal and distal ends. The proximal end of the irrigation supply line 439 is coupled to an irrigation source (not shown) and the distal end is coupled to means for flushing 435′. In the embodiment of FIG. 10, means for flushing 435′ preferably comprises a one-way valve electronically coupled to actuation means 419, although other mechanical or electrical valves may be employed.

In operation, when an obstruction is detected in cutting edge 12, means for interrupting 425′ is actuated to interrupt aspiration by compressing tubing section 22b′, for example, using any of the techniques described hereinabove. In a next step, means for flushing 435′ is actuated. Specifically, in a preferred embodiment, an electronic signal is sent to means for interrupting 435′ to cause a one-way valve to open. The opening of the one-way valve permits fluid in irrigation line 439 to be advanced through the valve and towards tubing section 22c′. Irrigation fluid introduced through tubing section 22c′ then imposes pressure upon cutting edge 12 to flush tissue obstructions away from the cutting instrument.

As will be apparent to one skilled in the art, the pressure of the irrigation fluid introduced into tubing 22c′ may be adjusted to facilitate removal of the obstruction. Further, means for interrupting 425′ and means for flushing 435′ may be actuated in a pre-programmed sequence using a microprocessor, as described hereinabove with respect to FIG. 9, or may be actuated in sequence mechanically using a multi-position switch, as described hereinabove.

It should be noted that, in the embodiment of FIG. 10, three tubing sections 22a′-22c′ are employed. Tubing section 22a′ is sealingly coupled to tubing section 22b′, while tubing section 22b′ is sealingly coupled to tubing section 22c′. In this embodiment, tubing section 22b′ may comprises a more flexible material than sections 22a′ and 22c′ to facilitate compression of section 22b′.

It will be apparent to one skilled in the art that illustrative components of apparatus 400 may be interchanged with components of apparatus 400′, and vice versa. For example, apparatus 400 of FIG. 9 may comprise three separate tubing sections, as depicted in FIG. 10. Further, means for interrupting 425 of FIG. 9 may be disposed circumferentially about aspiration tubing, as depicted in FIG. 10, and so forth.

It will also be apparent to one skilled in the art that tissue obstructions may be detected by the physician, who then actuates the apparatus described hereinabove, or alternatively, the tissue obstructions may be detected using one or more sensors coupled to the cutting instrument. In the latter case, the sensors may detect a tissue obstruction directly, or may determine that an obstruction is present based on a reduction in flow likely to be associated with an obstruction. Once an obstruction is suspected, the sensors may relay a signal to automatically actuate the means for interrupting and/or means for flushing to facilitate removal of the obstruction with little or no physician intervention.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. Apparatus suitable for clearing an obstruction from a cutting instrument during a surgical procedure, the apparatus comprising:
means for interrupting configured to interrupt aspiration flow in tubing coupled to the cutting instrument; and
means for flushing configured to flush fluid in the tubing, situated distal to the means for interrupting, in a distal direction,
whereby flushing the fluid in a distal direction facilitates removal of the obstruction.

2. The apparatus of claim 1 further comprising an actuation means having an exterior surface, the exterior surface enclosing the means for interrupting and the means for flushing.

3. The apparatus of claim 2 wherein the exterior surface is bulb-shaped.

4. The apparatus of claim 2 wherein the means for interrupting comprises at least one inward protrusion configured to apply compression to a section of the tubing.

5. The apparatus of claim 4 wherein the means for flushing comprises at least one fluid chamber disposed within the exterior surface and distal to the means for interrupting.

6. The apparatus of claim 5 wherein the fluid chamber comprises air, wherein compression of the fluid chamber is configured to cause compression of the tubing.

7. The apparatus of claim 5 wherein the fluid chamber comprises a liquid, wherein compression of the fluid chamber is configured to cause compression of the tubing.

8. The apparatus of claim 2 wherein the means for flushing comprises an interior compression member configured to selectively compress a section of the tubing distal to the means for interrupting.

9. The apparatus of claim 2 wherein the means for interrupting comprises at least one roller disposed within the exterior surface of the actuation means, wherein the roller is configured to selectively apply compression to a section of the tubing.

10. The apparatus of claim 9 wherein the means for interrupting further comprises at least one spring having proximal and distal ends, wherein the roller is coupled to the distal end of the spring.

11. The apparatus of claim 10 wherein the proximal end of the spring is fixedly attached to a proximal region of the actuation means.

12. The apparatus of claim 9 further comprising at least one roller guide disposed within the exterior surface and configured to guide movement of the roller.

13. The apparatus of claim 1 wherein the means for flushing comprises at least one roller configured to be advanced in a distal direction with respect to the tubing.

14. The apparatus of claim 1 wherein the means for interrupting comprises first and second rollers coupled to first and second handles, respectively, wherein actuation of the first and second handles causes the first and second rollers to selectively apply compression to a section of the tubing.

15. The apparatus of claim 14 wherein the first and second rollers also are the means for flushing.

16. The apparatus of claim 14 further comprising a first channel disposed in the first handle and a second channel disposed in the second handle, wherein the first and second channels are configured to guide movement of the first and second rollers, respectively.

17. The apparatus of claim 1 wherein the means for interrupting comprises at least one roller coupled to a roller axle, wherein the roller axle is configured for movement within a channel of a housing, and wherein the roller is configured to selectively apply compression to a section of the tubing.

18. The apparatus of claim 17 wherein the roller also is the means for flushing.

19. The apparatus of claim 18 wherein the channel comprises an advancement channel and a return channel.

20. Apparatus suitable for clearing an obstruction from a cutting instrument during a surgical procedure, the apparatus comprising:
an actuation means having an exterior surface configured to surroundingly engage a section of tubing coupled to the cutting instrument; and
at least one inward protrusion disposed within the exterior surface, the inward protrusion configured to selectively compress an outer portion of the tubing,
whereby compression of the actuation means is configured to cause the inward protrusion to compress the tubing to interrupt aspiration flow in the tubing, and whereby further compression of the actuation means is configured to cause fluid in the tubing to be flushed in a distal direction to facilitate removal of the obstruction.

21. The apparatus of claim 20 further comprising at least one fluid chamber disposed distal to the inward protrusion, wherein compression of the actuation means is configured to cause fluid in the fluid chamber to apply a compressive force upon the tubing.

22. The apparatus of claim 20 further comprising at least one interior compression member disposed distal to the inward protrusion and configured to selectively apply a compressive force upon the tubing.

23. Apparatus suitable for clearing an obstruction from a cutting instrument during a surgical procedure, the apparatus comprising:
an actuation means having an exterior surface configured to surroundingly engage a section of tubing coupled to the cutting instrument;
at least one spring having proximal and distal ends disposed within the exterior surface of the actuation means; and
at least one roller coupled to the distal end of the spring and configured to selectively engage an outer portion of the tubing,
whereby actuation of the actuation means is configured to cause the roller to compress the tubing to interrupt flow in the tubing, and further configured to flush fluid in the tubing in a distal direction to facilitate removal of the obstruction.

24. The apparatus of claim 23 wherein the proximal end of the spring is fixedly attached to a proximal region of the actuation means.

25. The apparatus of claim 23 further comprising at least one roller guide disposed within the exterior surface and configured to guide movement of the roller.

26. The apparatus of claim 25 wherein the roller guide has a proximal region, a distal region, and a slant disposed therebetween.

27. The apparatus of claim 26 wherein the proximal region of the roller guide is configured to house the roller so that the roller does not impose a substantial force upon the tubing.

28. The apparatus of claim 27 wherein the spring is configured to return the roller to the proximal region of the roller guide when no substantial external forces are applied to the actuation means.

29. The apparatus of claim 28 wherein the distal region of the roller guide is configured to cause the roller to apply a compressive force upon the tubing when the roller is advanced within the distal region.

30. Apparatus suitable for clearing an obstruction from a cutting instrument during a surgical procedure, the apparatus comprising:
a housing having at least one channel disposed therein, the housing configured to receive a section of tubing coupled to the cutting instrument; and
at least one roller coupled to a roller axle, the roller axle configured for movement within the channel,
wherein the roller is configured to be actuated to selectively compress an outer portion of the tubing to interrupt aspiration flow in the tubing, and further configured to flush fluid in the tubing in a distal direction to facilitate removal of the obstruction.

31. The apparatus of claim 30 wherein the housing has a lower surface, wherein a section of the tubing is configured to be disposed atop the lower surface.

32. The apparatus of claim 30 wherein the channel has a proximal region and a distal region separated by a central region having a contoured shape.

33. The apparatus of claim 32 wherein the proximal region of the channel is configured to house the roller axle in a fully aspirating state, so that the roller does not impose a substantial force upon the tubing.

34. The apparatus of claim 33 wherein the distal region of the channel is configured to cause the roller to apply a compressive force upon the tubing when the roller is advanced within the distal region.

35. The apparatus of claim 30 wherein the channel comprises an advancement channel and a return channel separated by a partition.

36. The apparatus of claim 35 wherein the advancement channel is configured to cause the roller to apply a compressive force upon the tubing when the roller is advanced within the advancement channel.

37. Apparatus suitable for clearing an obstruction from a cutting instrument during a surgical procedure, the apparatus comprising:
a handle configured for use with the cutting instrument;
means for interrupting disposed within the handle; and
means for flushing disposed within the handle at a location distal to the means for interrupting,
wherein the means for interrupting is configured to interrupt aspiration flow within the handle, and the means for flushing is configured to flush fluid, distal to the means for interrupting, in a distal direction to facilitate removal of the obstruction.

38. The apparatus of claim 37 further comprising electrical supply means coupled to the means for interrupting and the means for flushing.

39. The apparatus of claim 37 further comprising at least one section of aspiration tubing disposed within the handle.

40. The apparatus of claim 39 wherein the means for interrupting is disposed adjacent to a section of the aspiration tubing.

41. The apparatus of claim 39 wherein the means for interrupting fully encircles a section of the aspiration tubing.

42. The apparatus of claim 39 further comprising an irrigation supply line having proximal and distal ends, wherein the distal end of the irrigation supply line is coupled to the means for flushing.

43. The apparatus of claim 42 wherein the means for flushing comprises a one-way valve configured to selectively permit fluid communication between the irrigation supply line and the aspiration tubing.

44. The apparatus of claim 39 wherein a plurality of tubing sections are provided, and wherein at least one section of tubing is more flexible with respect to the other sections of tubing.

45. The apparatus of claim 39 further comprising an actuation means configured to actuate the means for interrupting and the means for flushing.

46. The apparatus of claim 45 wherein the actuation means is configured to electronically actuate the means for interrupting and means for flushing in a sequential manner using a predetermined routine.

47. The apparatus of claim 45 wherein the actuation means is configured to mechanically actuate the means for interrupting and means for flushing in a sequential manner.

48. A method for clearing an obstruction from a cutting instrument during a surgical procedure, the method comprising:
interrupting aspiration flow in tubing coupled to the cutting instrument; and
flushing fluid in the tubing in a distal direction,
whereby flushing fluid in a distal direction facilitates removal of the obstruction.

49. The method of claim 48 wherein interrupting aspiration flow comprises actuating a roller to apply a compressive force upon the tubing.

50. The method of claim 48 wherein actuating the roller comprises compressing an exterior surface of an actuation means, the actuation means having the roller disposed therein.

51. The method of claim 50 wherein flushing fluid in the tubing in a distal direction comprises applying further compression to the exterior surface of the actuation means to advance the roller in a distal direction.

52. The method of claim 48 wherein interrupting aspiration flow comprises compressing an actuation means to cause an inward protrusion, disposed within the actuation means, to apply a compressive force upon a first section of the tubing.

53. The method of claim 52 wherein flushing fluid in the tubing in a distal direction comprises applying further compression to the actuation means to compress a second section of the tubing, the second section disposed distal to the first section of the tubing.
